(19) United States
(12) Patent Application Publication
(10) Pub. No.: US 2019/0328815 A1
(43) Pub. Date: Oct. 31, 2019

(54) SKIN CARE TREATMENT

(71) Applicant: Laura FIELDS, Detroit, MI (US)

(72) Inventor: Laura FIELDS, Detroit, MI (US)

(21) Appl. No.: 16/271,285

(22) Filed: Feb. 8, 2019

Related U.S. Application Data
(60) Provisional application No. 62/663,631, filed on Apr. 27, 2018.

(51) Int. Cl. A61K 36/736; A61K 31/355; A61K 31/23; A61K 31/01; A61K 8/9789; A61K 8/92; A61K 8/67; A61K 8/31; A61K 8/34; A61Q 19/08; A61Q 19/00

(52) U.S. Cl. CPC A61K 36/736; A61K 31/355; A61K 31/23; A61K 31/01; A61K 8/9789; A61Q 19/007; A61K 8/678; A61K 8/31; A61K 8/345; A61Q 19/08; A61Q 19/00; A61K 8/922

(57) ABSTRACT

A skin care treatment composition including mineral oil, petroleum jelly, baby oil, vitamin E oil, lanolin cocoa butter, lotion, glycerin, and pure almond extract. A method of making the skin care treatment composition. A method of soothing skin, by applying the skin care treatment composition to skin of an individual, and soothing the skin of the individual. A method of reducing pain, by applying the skin care treatment composition to skin of an individual, and reducing pain in the individual. A method of treating eczema, by applying the skin care treatment composition to skin of an individual, and treating eczema in the individual. A method of lightening dark spots of the skin, by applying the skin care treatment composition to dark spots on skin of an individual. A method of removing bumps and calluses from the skin, by applying the skin care treatment composition to bumps and calluses.

### BACKGROUND OF THE INVENTION

### 1. Technical Field

The present invention relates to topical skin care treatments. In particular, the present invention relates to methods of treating pain.

### 2. Background Art

There are many different skin conditions or other conditions of the body that cause pain and irritation to individuals. Some skin conditions are chronic and must be managed by drugs and lifestyle changes, as they have no cure.

Pain is generally described as an unpleasant sensory experience in the body. Many people suffer from pain from medical treatments or medical conditions. For example, people undergoing chemotherapy can experience pain from headaches, muscles, stomach, or from nerve damage. While this pain can go away after treatment, nerve damage pain can get worse over time. This is usually treated with pain-relief medications.

Neuropathy, or peripheral neuropathy, is a nerve disorder that occurs when peripheral nerves such as sensory nerves, autonomic nerves, and motor nerves become damaged. The axon and myelin sheaths of the nerves degenerate. This damage can be caused by several different sources, such as autoimmune disorders, tumors, heredity, nutritional imbalances, infections, or toxins. One of the most common causes is diabetes. Some types of peripheral neuropathy have no known cause. Peripheral neuropathy is a relatively common disease, and at least 20 million Americans currently suffer from it. Symptoms include numbness, tingling, abnormal sensations, or pain in the feet and hands, and eventually in the arms and legs. This can affect movement and balance, detection of objects by the senses, and internal organs.

In treating peripheral neuropathy, there are generally two goals. The first goal is to treat the underlying cause, which can help the symptoms improve on their own. The second goal is to treat the symptoms causing pain. Common treatments include over-the-counter pain relief medications, such as nonsteroidal anti-inflammatory drugs (NSAIDS). Anti-seizure medications can be used, such as gabapentin, topiramate, pregabalin (LYRICA®, Pfizer, Inc.), carbamazepine, or phenytoin. Immunosuppressive medications can be used, such as prednisone, cyclosporine, or azathioprine. Capsaicin cream, lidocaine patches, and antidepressants are also used. Use of these treatments can result in unwanted side effects. In particular, pregabalin has had negative side effects of dizziness, drowsiness, dry mouth, edema, blurred vision, weight gain, difficulty concentrating, and increased risk of suicide.

Eczema is a skin condition in which the skin is inflamed or irritated. Eczema can be associated with allergic conditions, such as asthma and hay fever. Symptoms include itchy skin and dry, thickened, or scaly skin. Lotions and creams are used to keep the skin moist and relieve itching. Hydrocortisone 1% cream or other creams with corticosteroids can be used. Antihistamines, tar treatments, and phototherapy are also prescribed. ELIDEL® (pimecrolimus, Meda Pharma S.A.R.L.) and PROTOPIC® (tacrolimus, Astellas) are used in extreme cases, but also have a cancer risk associated with use.

Many individuals also suffer from under moisturized or dehydrated skin, which can be caused by inflammation, not drinking enough fluids, or simply being outside without protection on the skin. Moisturizers can be used to relieve dry skin, by rehydrating top layers of skin cells and sealing in water. Moisturizers can include humectants to attract moisture, occlusive to seal moisture within skin, and emollients. Moisturizers generally only hydrate skin or protect skin, but they do not necessarily have the ability to sooth skin. Some moisturizers do contain soothing ingredients, such as aloe vera or colloidal oats, but other soothing oils can be sold separately, such as calendula extract or chamomile oil.

Therefore, there remains a need for a skin care treatment that is effective in reducing pain as well as soothing skin without harmful side effects.

### SUMMARY OF THE INVENTION

The present invention provides for a skin care treatment composition including mineral oil, petroleum jelly, baby oil, vitamin E oil, lanolin cocoa butter, lotion, glycerin, and pure almond extract.

The present invention provides for a method of making the skin care treatment composition by mixing the mineral oil, baby oil, and vitamin E oil together in a bowl, adding the lanolin cocoa butter and mixing, adding the lotion and mixing, cooking the mixture on low heat for approximately five to six minutes, and settling with a lid on overnight.

The present invention provides for a method of soothing skin, by applying the skin care treatment composition to skin of an individual, and soothing the skin of the individual.

The present invention provides for a method of reducing pain, by applying the skin care treatment composition to skin of an individual, and reducing pain in the individual.

The present invention provides fora method of treating eczema, by applying the skin care treatment composition to skin of an individual, and treating eczema in the individual.

The present invention provides for a method of lightening dark spots of the skin, by applying the skin care treatment composition to dark spots on skin of an individual, and lightening the dark spots.

The present invention also provides for a method of removing bumps and calluses from the skin, by applying the skin care treatment composition to bumps and calluses on the skin of an individual, and removing the bumps and calluses.

### DETAILED DESCRIPTION OF THE INVENTION

The present invention generally provides for a skin care treatment composition that is used to soothe skin and reduce pain, as well as moisturize and protect skin. The formula of the composition is oil based and therefore moisturizes dry skin and protects the skin.

The term “soothe” or “soothing” as used herein, refers to the ability to reduce discomfort or pain in the body.

The skin care treatment composition includes mineral oil (16 oz.), petroleum jelly (such as VASELINE® (Unilever), 6 oz.), baby oil (such as JOHNSON'S®, mineral oil and perfume, 2 oz.), vitamin E oil (such as Target UP & UP™ vitamin E oil, 5 oz), lanolin cocoa butter (6 oz.), lotion (such as INTIMATE® with Vitamin E, 1.5 to 2 20 oz. bottles (30-40 oz. total), glycerin (3 caps full, or approximately 2 tablespoons), and pure almond extract (3 drops directly from a bottle or from a standard dropper). While representative amounts of each ingredient are given, these amounts can vary.

The skin care treatment composition is prepared by mixing all the oils together in a bowl, then adding the lanolin cocoa butter and mixing, and adding the lotion and mixing, cooking the resulting mixture on low heat (preferably in a pot on a stove) for approximately five to six minutes, and then settling with a lid on overnight.

The particular combination of components in the skin care treatment composition results in penetration of the composition into the skin to condition the skin and soothing of the skin in individuals experiencing pain. The skin care treatment composition is also protective of the skin and repels other compounds from entering the skin due to its oily nature. The skin care treatment can be applied to any area of the skin, such as, but not limited to, hands, feet, arms, legs, face, or torso.

The present invention provides for a method of soothing skin, by applying the skin care treatment composition to skin of an individual, and soothing the skin of the individual. In particular, the composition is able to soothe burning sensations in the skin caused by chemotherapy and neuropathy. The composition is able to provide relief to individuals. This method is evidenced by the Examples below.

The present invention provides for a method of treating pain, by applying the skin care treatment composition to skin of an individual, and treating pain in the individual. The pain can be caused by any condition, but the composition is especially useful in treating pain caused by chemotherapy and neuropathy. The composition reduces and/or eliminates pain in the body. This method is evidenced by the Examples below.

The present invention provides for a method of treating eczema, by applying the skin care treatment composition to skin of an individual, and treating eczema in the individual. The composition is able to reduce inflammation and irritation in the skin, and reduce itching due to eczema. This method is evidenced by the Examples below.

The present invention can also lighten dark spots of the skin, as well as remove bumps and calluses with use over a period of time. Therefore, the present invention provides for a method of lightening dark spots of the skin, by applying the skin care treatment composition to dark spots on skin of an individual, and lightening the dark spots. The present invention also provides for a method of removing bumps and calluses from the skin, by applying the skin care treatment composition to bumps and calluses on the skin of an individual, and removing the bumps and calluses.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

The composition of the present invention is able to penetrate skin due to the combination of moisturizers and oils used. The composition is also able to soothe skin due to the combination of ingredients or a particular ingredient. The Examples below demonstrate that soothing of the skin has clearly occurred.

### EXAMPLE 1

An individual used the skin care treatment composition after experiencing extremely dry skin from chemotherapy treatments. The skin care composition was the only treatment that worked to restore her skin.

### EXAMPLE 2

An individual works as a dishwasher and his hands are always in water. He had never been able to keep his hands soft. Using the skin care treatment composition restored the softness in his hands.

### EXAMPLE 3

An individual was in an accident that left her with uncomfortable side effects of burning tingly feet. The individual found that the skin care treatment composition was able to soothe her feet.

### EXAMPLE 4

An individual suffered from eczema on her neck and arms. After using the skin care treatment composition, the eczema was cleared from her neck and arms.

Each of the above Examples shows that individuals using the skin care treatment composition reacted favorably and the skin care treatment composition has the ability to moisturize, protect, and soothe skin and reduce pain. Individuals from ages of 3 to 100 have used the skin care treatment composition. Also, individuals with very sensitive skin with initial reluctance to use the skin care treatment composition did not have any adverse reactions from it and continue to use it successfully.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A skin care treatment composition comprising mineral oil, petroleum jelly, baby oil, vitamin E oil, lanolin cocoa butter, lotion, glycerin, and pure almond extract.

2. The skin care treatment composition of claim 1, wherein said skin care treatment composition is further defined as 16 oz. mineral oil, 6 oz. petroleum jelly, 2 oz. baby oil, 5 oz. vitamin E oil, 6 oz. lanolin cocoa butter, 30-40 oz. lotion, 2 Tablespoons glycerin, and 3 drops pure almond extract.

3. A method of making the skin care treatment composition of claim 1, including the steps of:
mixing the mineral oil, baby oil, and vitamin E oil together in a bowl;
adding the lanolin cocoa butter and mixing;
adding the lotion and mixing;
cooking the mixture on low heat for approximately five to six minutes; and
settling with a lid on overnight.

4. The method of claim 3, wherein said heating step is performed in a pot on a stove.

5. A method of soothing skin, including the steps of:
applying a skin care treatment composition including mineral oil, petroleum jelly, baby oil, vitamin E oil, lanolin cocoa butter, lotion, glycerin, and pure almond extract to skin of an individual; and
soothing the skin of the individual.

6. The method of claim 5, wherein said soothing step is further defined as soothing burning sensations in the skin caused by chemotherapy and neuropathy.

7. The method of claim 5, further including the step of restoring softness in the skin.

8. The method of claim 5, further including the step of moisturizing and protecting the skin.

9. The method of claim 5, wherein the skin is chosen from the group consisting of hands, feet, arms, legs, face, torso, or combinations thereof.

10. A method of reducing pain, including the steps of:
applying a skin care treatment composition including mineral oil, petroleum jelly, baby oil, vitamin E oil, lanolin cocoa butter, lotion, glycerin, and pure almond extract to skin of an individual; and
reducing pain in the individual.

11. The method of claim 10, wherein the pain is caused by chemotherapy and neuropathy.

12. A method of treating eczema, including the steps of:
applying a skin care treatment composition including mineral oil, petroleum jelly, baby oil, vitamin E oil, lanolin cocoa butter, lotion, glycerin, and pure almond extract to skin of an individual; and
treating eczema in the individual.

13. The method of claim 12, further including the steps of reducing inflammation and irritation in the skin, and reducing itching due to eczema.

14. A method of lightening dark spots of the skin, including the steps of:
applying a skin care treatment composition including mineral oil, petroleum jelly, baby oil, vitamin E oil, lanolin cocoa butter, lotion, glycerin, and pure almond extract to dark spots on skin of an individual; and
lightening the dark spots.

15. A method of removing bumps and calluses from the skin, including the steps of:
applying a skin care treatment composition including mineral oil, petroleum jelly, baby oil, vitamin E oil, lanolin cocoa butter, lotion, glycerin, and pure almond extract to bumps and calluses on the skin of an individual; and
removing the bumps and calluses.
